SEQUENCE LISTING (12 sequences)

SEQ ID NO: 1 - DNA, 601 bp, Homo sapiens
ggcttagaatgagggcccacactaagagaaaacagataaggcaacttacaaggctgttgaccagctttgtaaactgttcttcaagaaacaatcccaagcttttagctactataccatttttgatatttaatgagctgacgattaaaaatacttgtttgttgcctgttacttgaaaactataaatgccaatgcagtggactcagggtgccacattcttagttaattaagcacccccatttgctgcactaacgggtagcttttaatttgttccaaagggagctaagcagcaagccccggctgyctggagtgctgctatgaaattcctttggttggtgctctatggtcagtagggacggaagcagcaaatggcaacataatggagaaaacatctttaactcaggaagggaggggaggataacccttaagtgctctaaatgaataaaattcaaaagctaaaaacccacaccatgctccagccatatatggcaaagaagcaaaggcctggatacagagaggccggcagcggagcaggatccacaggcagcagctaggggcagccatgaaggtgatcagacagccaacttcctgaggggcctttggg

SEQ ID NO: 2 - DNA, 601 bp, Homo sapiens
taaccagaaaagattaaaagcagtgattaattcttttttaaaggaaagtccaaaggttctggctaaactgactttaaactaagaatcggggagctcattgctttcgctgctgcggccacagccatgagtatgctcaggtttcagatgaggctcgcctttagtgtcctctgctgtggcaagaagaaggtctggttggaccccaataagaccaatgaaatcgccaatgccaacttccatcagcagatccagaagctgatcaaagatgggctgatcatctacaaacctgtgactttccattccygggcttgatgccgggaaaacaccttggcccgctggaagggcaggcacatgggcataagtaagcgaaagggtacagccaatgcccaaatgccagggaacgtaacttggatgaggagaatgcggattctgtgctggctgctgagaagatactgtgaatctaagaagattgatcaccacacatatcacagcctgtacctgaaggtgaaggggaatgtgttcaaaaacaagtggattctcatggaacacatcctcaagctgaaggcagacaaggcccacaagaagctgcaggctgaccaggcta

SEQ ID NO: 3 - DNA, 707 bp, Homo sapiens
ttaaactttttcctttgtttaaattttttattttgaatttttgtgtaattttgaactgaatgtgaatttgagtgtgaatattatatatacaaatatgtgtatattatttatggggtacatgcaatgaaaatttccttttttttttttttttgagacggggtcttgctctgtctcccaggctgtagtgcagtggcatgatctcagctcaatgcagcctctgcctcctggttccagtgattctcctgcctcagcctcctgggtagctgggagtacaggcacgtgccaccacgccaggctaatttttgtatttttagtagagacagggtttcaccatgttggccaggctggccttgaactcctgacctcaggtgatccgcccacctcagccttcctccaaaagtgctaggattacaggtgtgagccacctcgcctggctgaaaacctttttgattacacaaatctctgagcaaataattatactgctagatcatttaaaatagctcctcyatattaattcctaagttttctgctgttattcaaactcaactcctggttttacaggaaaaatagaaaacatattattaggaccatttgaatcaaatgttttcatatgtttaaaacttaaaaaaaaaaatctggccctggaaaaaagtactgaaaagactgctagttcactgtcaggcttctgcataaagataccgagccgg

SEQ ID NO: 4 - DNA, 601 bp, Homo sapiens
gggacagaagcgcagtgcttttggcaccttgcccaagtcacaccctacataggatatcctcaaggaagcaacaggtggtagagagcacaggacagagaaactgaggttggttgatctggtgagtgaggtgcctagattggaggggcagattctggagagaaggctggcaggctctgagaggaggatgtggatgagtgactccaaagtgagctgggagatggggctcaggcctggatgccagtcacccacaattataggtgactcacatccagacagtgactgacaaaggggacctatctcracactctcatagcctcaggctcacctatgttgtttactagttggtccatggctggagaggaggctagtgaactgcattaacatggtcattctgagtaactcactcaggactggctagcaccttcctctatggcttcacacaggtatacaaggctacatctctacaaggctggtaattgacaaagctttacccccaaaggcggtctggtagagagactgtgggcgtggctgacaaaggagagaaaaatcagacatgtgtggccccttagaggacttcaccttctcagaccacttttaccta

SEQ ID NO: 5 - DNA, 845 bp, Homo sapiens
caacaaggggcaggccagggggtgctgccaagagaaagagagactgggagtacctgggtcctccaggatggcgcctgggaagcctcatagctcctactattcagttcccagaattaaaatatttggcttttaacctcaggagccactaaaaaaaacaggtatcattatatatctgtttcttgggacagggtctcgctctgttgcccaggctagagtgcagtggcacagtcatagctcactgcaggctcgacttcccaggcttaggccatcctcccacttcagccccctgaggtagctggggactacaggcatgcaccaccatgcctggctaattttaaaaaaaaattttttttgtagtcatggggtctcactatgttgcctaggctggtcttgaactcctaagctcaagcattcctcctgcctcagcctccctctcatgctgggattacaggcatgagacactgcatctggcccataattctattttcaatgataggakagtaaattcagggtcataaaggaagcctgaagtataagtaaggatataggtagggcttggtcatttacaaaagtcaccatgagacagccttctgaggagttggaaatgatccatagcttggtctgggtgaggattacacagttatatacttccataaaaattcactgagatgtacacttaagatttatgtactttacttttgtaaattttacatcgataaaaacttttaaaaaaaatcagctctgcctgtgcttaattggtctactaggtgcacttggctgcaagagccagttctgggcactctgctaactaaggtagcgctctgctaaccagggtgacccctgca

SEQ ID NO: 6 - DNA, 201 bp, Homo sapiens
gacacagcacacaaagagataccttgttgcttcttgcggcacatcacagtgaagagcgtcgcgaatgccgatatgactaccagtggcactgtgatggccayggctctgatgggcccggcccacggggagtgcactgggaaatttcaaagggaaagtttttaaacttaagcagtgtaaaggttcaaagagaaacaacttttt

SEQ ID NO: 7 - DNA, 601 bp, Homo sapiens
tactagagattttaaaccatggttatatatggaccatatgattacataaaaatatttgtttgaaatatttaatgaagagatcaaaaatcaattatatataaactttttttaaaagcatgttaagaaccatataaaaatataactgtgcacaccttcaatagcttgaaattttagtgttgtaaacagttggaattctatatgaactgggttcattctatagcattaatagggctttcttgtgttatgcatacctcaattatataatcccctggagaaacattttgaaggaggcagctggtcrtctctgtagtttgctcctgcaacagaccaaagaacactttaaaaactcacttcttgggcaaaggtcttcaggacacctcttctcccccagagctgaaggagacttgccaccagtctcaaccacagttttcagaaaatgtgattagatcgaaaagcaactgtgtttatctgccacatgttatctgaacttccctttgataaaaggcccaggacagcccaggacccttgccatagtctatgcttagctggtggcaccctcagcaggaatatttgatttgaagacaaagaaactggtttgaac

SEQ ID NO: 8 - DNA, 808 bp, Homo sapiens
ctgctcaggaggctgaggcatgagaatcgcttgaacccaggaggtggaggttgcagtgagcagagatcacgccactacactccagcctgggtgacagagtgagactctatctcaaaaaagaaaaagaaaagaaaagaaaaaagaactgatagatggggtactgcggttggtgcccaggcctggtcagcgatggccaaccaactgcagactgcagatccctggcccatctggaacccagattcacacagattcaccttgtgtgaatttcacacagattcaccttgtgtgaatttcacacagattcactctgtgtgaaattttaaaaataactgccattattaaaaaatgtttacacaaaaagcacaaatttctcactattcttggaaatcaatttgctaccttcagtgggaattcctcatggccacyatatttggaattaaggagtcaaggctcccctcagactagacatgagctatccagtttgccatactgtccaccattccctagcaccctgcactggggcaactttgtttatgtgttccttgctgggccatactggcagctatgtccttaacaacacatgcagataaaccccaaggaacacagggaaagtctccttacacaacttttctgaacactatggaagacacttgacctggtcaagctcccaaatacttgagtggcctaattgagactgagctgctttagctgttgacaaacctgaacaggttcagaaaattcgtcaaccccaagactgcgaatccttgataagcatggctatgtaatctcttagatgactcaaagggct

SEQ ID NO: 9 - DNA, 704 bp, Homo sapiens
aggtcccctcccctggtgggctgcttgccgcatcacactgtgtgttcctgacacatcggtctgactgctactcacttgaggggaacatggtaattcattcccacaatttgagtagcatggcacttttagggatgtagatgcaaaatggtagttccaattccacagaaaatcagagatcataagaccccgtggccatccacrccgggtcatggagctcagaagcaggagaaaacatcccaggtattagttcttacatgcattttaaagaccttttggctgcgcgtggtggctcacacctgtaatctcagcactttgggaggccgaggcaggtggatcacctgaagtcaggagtttgagaccagcctgaccaacatggtgaaaccccatctctactaaaaatacaaaaaattaactgggtgtaatggcgggcacctgtaatcccagctacttgggaggctgaggcgggagaatcgcttgaacccgggaggcggaggttgcagtgagccgagatcgcgccatcgtactccatcctgggtgacaagaacaaaactccatctcaaacaaacaaacaaacaaaaacctttcattcctggaattcctcaagtgcacagcttctcagcacatgcatcacagatgctctataaggctgtccttagctcaacgcccaaataatacttcagctgctctagagccctcagaaacca

SEQ ID NO: 10 - DNA, 601 bp, Homo sapiens
ggctttaagaaagaacttttttaaaagctggattcacttctgaatctttaaaataatcccccttaatcaaatctcattttcaccaaataactactaccaacattagtggtaagtttcaactacacttagtccagtggtttttcaaaaaccaatgtgccaaacccaagagttccaatgactgagaagccagagacggctagtgatgactgcctcaggaggagaagaaatgttcgtaggaaaattcatagcaaagggacagaggaaaagaacaagggggcgagaaggtagcctagagatgaayggtctatgaaagaaatgggaggaagcctgagaagaaatatggtggaaaagtgtatagtaaatgccaatgatcagactcatcgttagcaatatgtatgcagagaagtgccatggaaagagaggcgagaagcacatagtgtgaggttggaacttcaggcagcccagttctgcctggatggtcagaacttggaatgcttgagattggcctctgattcaactggaccgattacatttgctcagaaatgtactggctcactccagggatggaggtgagtattctcagagcaagatgcatctgtga

SEQ ID NO: 11 - DNA, 601 bp, Homo sapiens
accttatccagaactgctgcatcgtgactcatctttgttgaaggaaaatgctgtgcgattcgaattgagctactgattggcatgctcataagaggtttcctggtgaactgcactctttatttctcaggactcacatcctcttcactacacctcttccagttgaggatagaaagttttggaattatgaatctgaatgagcacaaaaccagaagagtaagaataacaattataaagtcctctgatggggttcacatgatgggttttgatcattgcacacttcttcagttttaagaaatgagtkatttcttcttctttctcctccctgtctctctcacacacacataaaacgtctggtataagaagagactactataaatagcctggtgaatcatgatcaggctcttcactttgaaactaacactgcagctcaaaatttattttaaaatgaatctactttcaatagcaagacaattctcaaagcaggacatttagaacagccataatttgtgaatattatagaagaacccagcctaattatttccttttcacgagcaagtgactggttaaaaccaaaattcctcctctcatgtaaaggtttctc

SEQ ID NO: 12 - DNA, 644 bp, Homo sapiens
aagatcagttacagtgcaggctaaactgccttaaaaaaaaaaagaggacccaaagcacagtggcttaaataatactttatttatcttccatataacagccctgaggtaaggtatccagactggtgggcagctgtgctccatagttatttgtggactcagttttcttccatctttttggcataccatcccttaggtattgtcctcatctgcatgatctctatgagatcacagacacttctgctcatatttctttggagaaaacctagtcacatggccacatctcttacttcaaaaggttagacaaattgtaaagcatacttttaaaaaaaaaaaaaaatcaaagcttatgaaagccaagtctttaaaaatgtttcatattgttcatgaactttaaaaaaaataagtcacatgcatttataagaaatgtttttaggatttttcctkctacatcataaaagacctgtatttcctcttattgccaaatacaatcactgttaatagatatttcagatatttccctataatacttttttcttggcatttttaacatggttgtgttcaagctatttataccattttgaatcccgtctttttttcaactgttcacataatttacctgtcattacaaactctttgtaaatatt

(19) United States
(12) Patent Application Publication
(10) Pub. No.: US 2011/0177969 A1
(43) Pub. Date: Jul. 21, 2011

(54) THE ROLE OF IL17RD AND THE IL23-1L17 PATHWAY IN CROHN'S DISEASE

(75) Inventors: Jerome I. Rotter, Los Angeles, CA (US); Kent D. Taylor, Ventura, CA (US); Stephan R. Targan, Santa Monica, CA (US); Dermot P. McGovern, Los Angeles, CA (US); Ling Mei, Pasadena, CA (US)

(73) Assignee: CEDARS-SINAI MEDICAL CENTER, Los Angeles, CA (US)

(21) Appl. No.: 13/121,929

(22) PCT Filed: Oct. 1, 2009
(86) PCT No.: PCT/US09/59190

§ 371 Date: Mar. 30, 2011

(30) Foreign Application Priority Data
Oct. 1, 2008 (US) 61/101779

(51) Int. Cl. C12Q 1/68; C40B 30/04

(52) U.S. Cl. 506/9; 435/6.11

(57) ABSTRACT

The present invention relates to methods of diagnosing susceptibility to Crohn's Diseaese by determining the presence or absence of susceptibility variants at the IL17RD locus. in one embodiment, the present invention provides a method of diagnosing and/or predicting susceptibility to Crohn's Disease by determining the presence or absence of an interaction between IL17RD Block 2 Haplotype 2 and IL23R Block 2 Haplotype 2 and/or IL12RB2 Haplotype 4, where the presence of an interaction between IL17RD Block 2 Haplotype 2 and IL23R Block 2 Haplotype 2 and/or IL12RB2 Haplotype 4 is indicative of susceptibility to Crohn's Disease.

### FIELD OF THE INVENTION

The invention relates generally to the fields of inflammation and autoimmunity and autoimmune diseases and, more specifically, to methods for diagnosing and predicting disease progression of Crohn's disease.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Crohn's disease (CD) and ulcerative colitis (UC), the two common forms of idiopathic inflammatory bowel disease (IBD), are chronic, relapsing inflammatory disorders of the gastrointestinal tract. Each has a peak age of onset in the second to fourth decades of life and prevalences in European ancestry populations that average approximately 100-150 per 100,000 (D. K. Podolsky, N Engl J Med 347, 417 (2002); E. V. Loftus, Jr., Gastroenterology 126, 1504 (2004)). Although the precise etiology of IBD remains to be elucidated, a widely accepted hypothesis is that ubiquitous, commensal intestinal bacteria trigger an inappropriate, overactive, and ongoing mucosal immune response that mediates intestinal tissue damage in genetically susceptible individuals (D. K. Podolsky, N Engl J Med 347, 417 (2002)). Genetic factors play an important role in IBD pathogenesis, as evidenced by the increased rates of IBD in Ashkenazi

Jews, familial aggregation of IBD, and increased concordance for IBD in monozygotic compared to dizygotic twin pairs (S. Vermeire, P. Rutgeerts, Genes Immun 6, 637 (2005)). Moreover, genetic analyses have linked IBD to specific genetic variants, especially CARD15 variants on chromosome 16q12 and the IBD5 haplotype (spanning the organic cation transporters, SLC22A4 and SLC22A5, and other genes) on chromosome 5q31 (S. Vermeire, P. Rutgeerts, Genes Immun 6, 637 (2005); J. P. Hugot et al., Nature 411, 599 (2001); Y. Ogura et al., Nature 411, 603 (2001); J. D. Rioux et al., Nat Genet 29, 223 (2001); V. D. Peltekova et al., Nat Genet 36, 471 (2004)). CD and UC are thought to be related disorders that share some genetic susceptibility loci but differ at others.

The replicated associations between CD and variants in CARD15 and the IBD5 haplotype do not fully explain the genetic risk for CD. Thus, there is need in the art to determine other genes, allelic variants and/or haplotypes that may assist in explaining the genetic risk, diagnosing, and/or predicting susceptibility for or protection against inflammatory bowel disease including but not limited to CD and/or UC.

### SUMMARY OF THE INVENTION

Various embodiments include a method for diagnosing susceptibility to Crohn's disease in an individual, comprising determining the presence or absence of a risk haplotype at the IL17RD genetic locus in the individual, and diagnosing susceptiblity to Crohn's disease in the individual based upon the presence of the risk haplotype at the IL17RD genetic locus. In another embodiment, the risk haplotype at the IL17RD genetic locus comprises IL17RD Block 2 Haplotype 2. In another embodiment, the risk haplotype at the IL17RD genetic locus comprises SEQ. ID. NO. 1, SEQ. ID. NO. 2 and/or SEQ. ID. NO. 3.

Other embodiments include a method for diagnosing susceptibility to Crohn's disease in an individual, comprising obtaining a sample from the individual, assaying the sample to determine the presence or absence of a risk haplotype at the IL17RD genetic locus in the individual, and diagnosing susceptibility to Crohn's Disease in the individual based upon the presence of the risk haplotype at the IL17RD genetic locus in the sample. In another embodiment, the risk haplotype at the IL17RD genetic locus comprises IL17RD Block 2 Haplotype 2. In another embodiment, assaying the sample comprises genotyping for one or more single nucleotide polymorphisms.

Other embodiments include a method of determining a low probability of developing Crohn's disease in an individual, relative to a healthy subject, comprising obtaining a sample from the individual, assaying the sample to determine the presence or absence of one or more protective haplotypes at the ILI7RD genetic locus in the individual, and diagnosing a low probability of developing Crohn's disease in the individual, relative to a healthy subject, based upon the presence of one or more protective haplotypes at the IL17RD genetic locus. In another embodiment, the one or more protective haplotypes at the IL17RD genetic locus comprises I7RD Block 1 Haplotype 2 and/or ILI7RD Block 2 Haplotype 3. In another embodiment, the one or more protective haplotypes at the IL17RD genetic locus comprises SEQ. ID. NO.: 4, SEQ. ID. NO.: 5, SEQ. ID. NO.: 6, SEQ. ID. NO.: 7, SEQ. ID. NO.: 8, and/or SEQ. ID. NO.: 9. In another embodiment, the one or more protective haplotypes at the ILI7RD genetic locus comprises SEQ. ID. NO.: 1, SEQ. ID. NO.: 2 and/or SEQ. ID. NO.: 3. In another embodiment, assaying the sample comprises genotyping for one or more single nucleotide polymorphisms. In another embodiment, assaying the sample comprises specific hybridization of genomic DNA to arrayed probes.

Other embodiments include a method of diagnosing susceptibility to Crohn's disease in an individual, comprising obtaining a sample from the individual, assaying the sample for the presence or absence in the individual of a risk haplotype at the IL17RD genetic locus, a risk haplotype at the IL23R genetic locus, and a risk haplotype at the IL12RB2 genetic locus, and diagnosing susceptiblity to Crohn's disease in the individual based upon the presence of the risk haplotype at the IL17RD genetic locus, the risk haplotype at the IL23R genetic locus, and the risk haplotype at the IL12RB2 genetic locus. In another embodiment, the risk haplotype at the IL23R genetic locus comprises IL23R Block 2 Haplotype 2. In another embodiment, the risk haplotype at the IL12RB2 genetic locus comprises IL12RB2 Haplotype 4. In another embodiment, the risk haplotype at the IL12RB2 genetic locus comprises SEQ. ID. NO.: 10, SEQ. ID. NO.: 11 and/or SEQ. ID. NO.: 12. In another embodiment, assaying the sample comprises performing a whole-genome microarray assay. In another embodiment, assaying the sample comprises multidimensionality reduction.

Other features and advantages of the invention will become apparent from the following detailed description, taken in conjunction with the accompanying drawings, which illustrate, by way of example, various embodiments of the invention.

### BRIEF DESCRIPTION OF THE FIGURES

Exemplary embodiments are illustrated in referenced figures. it is intended that the embodiments and figures disclosed herein are to be considered illustrative rather than restrictive.

FIG. 1 depicts, in accordance with an embodiment herein, association of IL17-IL23 pathway-related haplotypes with Crohn's Disease.

FIG. 2 depicts, in accordance with an embodiment herein, interaction between IL23R risk haplotypes and IL17A risk haplotype in non-Jewish subjects.

FIG. 3 depicts, in accordance with an embodiment herein, IL17RD haplotypes.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

“Haplotype” as used herein refers to a set of single nucleotide polymorphisms (SNPs) on a gene or chromatid that are statistically associated.

“Risk” as used herein refers to an increase in susceptibility to IBD, including but not limited to CD and UC.

“Protective” and “protection” as used herein refer to a decrease in susceptibility to IBD, including but not limited to CD and UC.

“CD” and “UC” as used herein refer to Crohn's Disease and Ulcerative colitis, respectively.

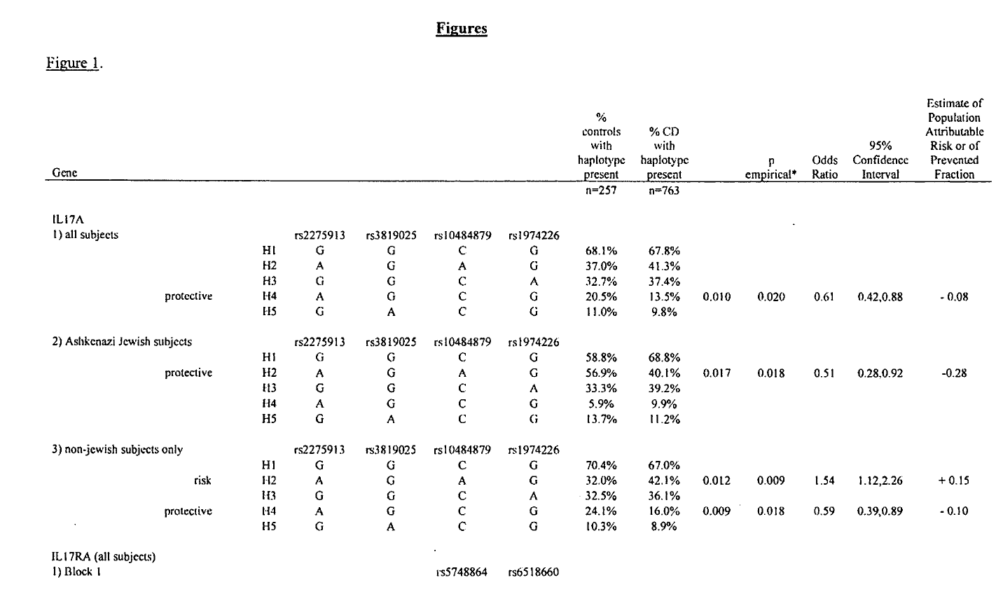

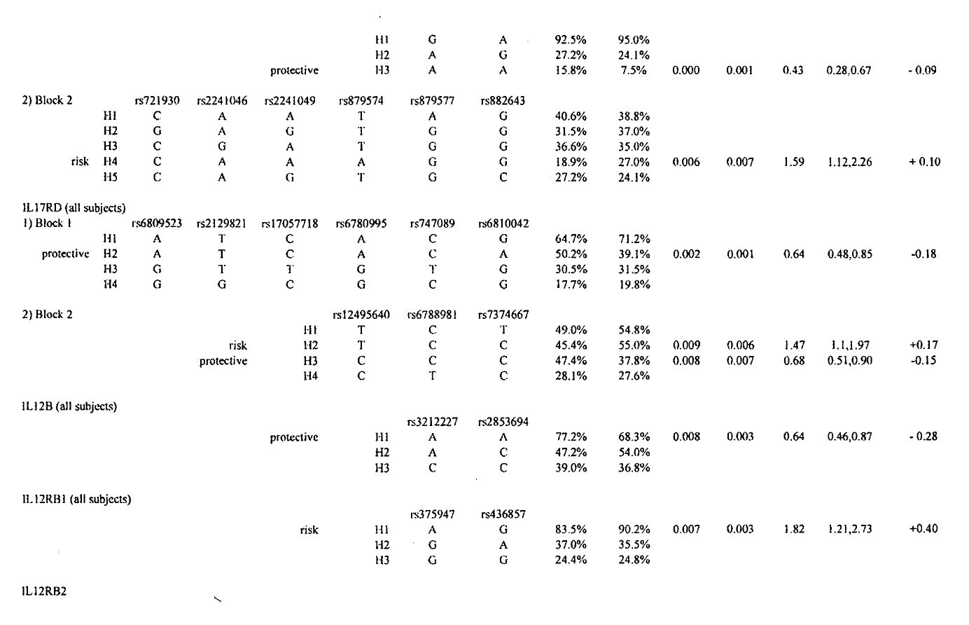

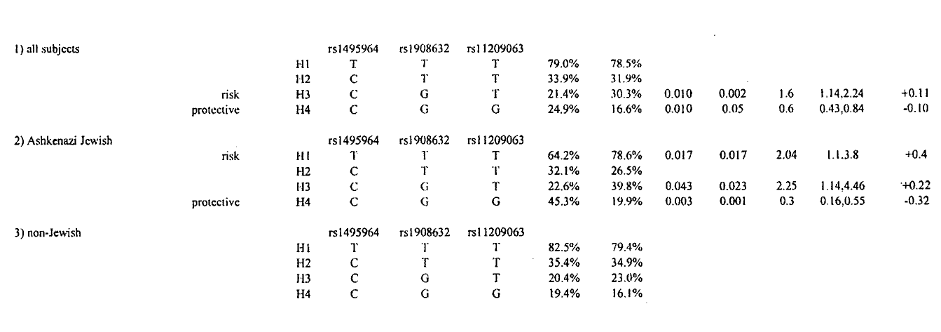

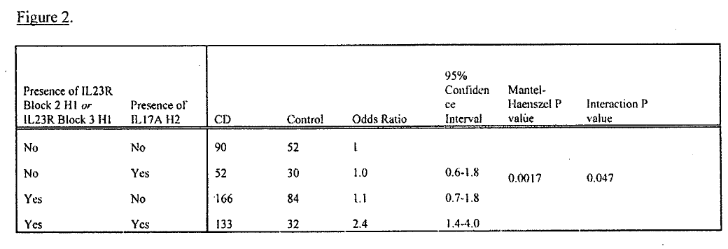

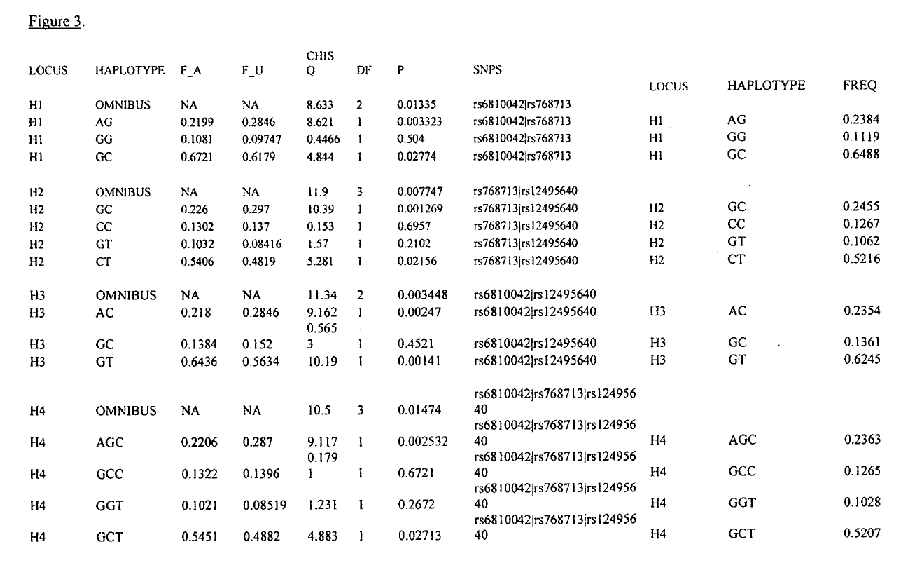

As used herein, the abbreviation “B” designates a haplotype block and “H” designates a haplotype. For example, “IL17RD B2H2” refers to Block 2 Haplotype 2 at the IL17RD genetic locus. Similarly, “IL17RD B1H2” and “IL17RD B2H3” refers to Block 1 Haplotype 2 and Block 2 Haplotype 3, respectively, at the IL17RD genetic locus. Although in no way limited, various SNPs and alleles described in FIG. 1 herein may he used to describe the various haplotypes referenced herein. For example. Block 2 at the IL17RD genetic locus includes SNPs rs12495640, rs6788981, and rs7374667, described herein as SEQ. ID. NO.: 1, SEQ. ID. NO.: 2 and SEQ. ID. NO.: 3, respectively. Similarly, Block 1 at the IL17RD genetic locus includes SNPs rs6809523, rs2129821, rs17057718, rs6780995, rs747089, and rs6810042, described herein as SEQ. ID. NO.: 4, SEQ. I.D. NO.: 5, SEQ. ID. NO.: 6, SEQ. ID. NO.: 7, SEQ. ID. NO.: 8, and SEQ. ID. NO.: 9, respectively. Similarly, risk haplotype H4 at the IL12RB2 genetic locus includes SNPs rs 1495964, rs 1908632, and rs 11209063, described herein as SEQ. ID. NO.: 10, SEQ. ID. NO: 11 and SEQ. ID. NO.: 12, respectively.

As used herein, the term “biological sample” means any biological material from which nucleic acid molecules can be prepared. As non-limiting examples, the term material encompasses whole blood, plasma, saliva, cheek swab, or other bodily fluid or tissue that contains nucleic acid.

The inventors performed a genome-wide association study testing autosomal single nucleotide polymorphisms (SNPs) on the Illumina HumanHap300 Genotyping BeadChip. Based on these studies, the inventors found single nucleotide polymorphisms (SNPs) and haplotypes that are associated with increased or decreased risk for inflammatory bowel disease, including but not limited to CD. These SNPs and haplotypes are suitable for genetic testing to identify at risk individuals and those with increased risk for complications associated with serum expression of Anti-Saccharomyces cerevisiae antibody, and antibodies to 12, OmpC, and Cbir. The detection of protective and risk SNPs and/or haplotypes may be used to identify at risk individuals predict disease course and suggest the right therapy for individual patients. Additionally, the inventors have found both protective and risk allelic variants for Crohn's Disease and Ulcerative Colitis.

Based on these findings, embodiments of the present invention provide for methods of diagnosing and/or predicting susceptibility for or protection against inflammatory bowel disease including but not limited to Crohn's Disease and ulcerative colitis. Other embodiments provide for methods of proposing inflammatory bowel disease including but not limited to Crohn's Disease and ulcerative colitis. Other embodiments provide for methods of treating inflammatory bowel disease including but not limited to Crohn's Disease and ulcerative colitis.

The methods may include the steps of obtaining a biological sample containing nucleic acid from the individual and determining the presence or absence of a SNP and/or a haplotype in the biological sample. The methods may further include correlating the presence or absence of the SNP and/or the haplotype to a genetic risk, a susceptibility for inflammatory bowel disease including but not limited to Crohn's Disease and ulcerative colitis, as described herein. The methods may also further include recording whether a genetic risk, susceptibility for inflammatory bowel disease including but not limited to Crohn's Disease and ulcerative colitis exists in the individual. The methods may also further include a prognosis of inflammatory bowel disease based upon the presence or absence of the SNP and/or haplotype. The methods may also further include a treatment of inflammatory bowel disease based upon the presence or absence of the SNP and/or haplotype.

In one embodiment, a method of the invention is practiced with whole blood, which can be obtained readily by non-invasive means and used to prepare genomic DNA, for example, for enzymatic amplification or automated sequencing. In another embodiment, a method of the invention is practiced with tissue obtained from an individual such as tissue obtained during surgery or biopsy procedures.

As disclosed herein, the inventors have determined that IL17RD is associated with CD and that there is a gene-gene interaction within IL23-IL17 pathway genes. 763 CD subjects and 254 controls were genotyped for single nucleotide polymorphisms in the IL23A, IL23R, IL17A, IL17RA, IL12B, IL12RB1, IL12RB2 and IL17RD genes using Illumina and ABI platforms. Haplotypes were assigned using Phase v2 and were tested for association with CD by chi square test. The inventors utilized multidimensionality reduction (MDR) to explore gene-gene interactions.

As further disclosed herein, two Blocks (B) of IL17RD were associated with CD. CD patients had a higher frequency of haplotype2 in block2 (B2H2, 55.0% vs. 45.4%, OR=1.5, p=0.01) and a lower frequency of B1H2 (39.1% vs. 50.2%, OR=0.64, p=0.002) and B2H3 (37.8% vs. 47.4%, OR=0.68, p=0.01) when compared with controls. Haplotypes with increased risk for CD were observed in the IL23R_B2H1 and B3H1, IL17A_H2, IL17RA_B2H4, IL12RB1_H1 and IL12RB2_H3; haplotypes with decreased risk were observed in the IL23R_B2H2 and B3H2, IL17A_H4, IL17RA_B1H3, IL12B_H1 and IL12RB2_H4. MDR analysis suggested interaction between IL23R_B2H2, IL12RB2_H4 and IL17RD_B2H2 (CV consistency 10/10. tested accuracy 59.7%. p=0.002). The following logistic regression analysis confirmed the interaction (IL23R_B2H2*IL12RB2_H4, p<0.0001; IL23R_B2H2*IL17RD_B2H2, p=0.02). Thus, the inventors have found IL17RD to he significantly associated with CD and likely to interact with IL23R in the risk of developing CD.

In one embodiment, the present invention provides a method of diagnosing and/or predicting susceptibility to Crohn's Disease by determining the presence or absence of a risk haplotype and/or variant at the IL17RD locus, where the presence of the risk haplotype and/or variant at the IL17RD locus is indicative of susceptibility to Crohn's Disease. In another embodiment, the present invention provides a method of treating Crohn's Disease by determining the presence of a risk haplotype and/or variant at the IL17RD locus and treating the Crohn's Disease. In another embodiment, the risk haplotype at the IL17RD locus is IL17RD Block 2 Haplotype 2. In another embodiment, the present invention provides a method of diagnosing and/or predicting susceptibility to Crohn's Disease by determining the presence or absence of an interaction between IL17RD Block 2 Haplotype 2 and IL23R Block 2 Haplotype 2 and/or IL12RB2 Haplotype 4, where the presence of an interaction between IL7RD Block 2 Haplotype 2 and IL23R Block 2 Haplotype 2 and/or IL12RB2 Haplotype 4 is indicative of susceptibility to Crohn's Disease.

In another embodiment, the present invention provides a method of diagnosing and/or predicting protection against Crohn's Disease by determining the presence or absence of a protective haplotype at the IL17RD locus, where the presence of the protective haplotype at the IL17RD locus is indicative of a decreased likelihood of susceptibility to Crohn's Disease relative to a healthy individual. In another embodiment, the present invention provides a method of diagnosing and/or predicting protection against Crohn's Disease by determining the presence or absence of a protective variant at the IL17RD locus, where the presence of the protective variant at the IL17RD locus is indicative of a decreased likelihood of susceptibility to Crohn's Disease relative to a healthy individual. In another embodiment, the protective haplotype at the IL17RD locus is IL17RD Block 1 Haplotype 2. In another embodiment, the protective haplotype at the IL17RD locus is IL17RD Block 2 Haplotype 3.

### Variety of Methods and Materials for Assaying Samples to Determine the Presence or Absence of Variant Alleles

A variety of methods can be used to determine the presence or absence of a variant allele or haplotype. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The presence or absence of a variant allele or haplotype may involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhauser, Boston, (1994)).

A TaqmanB allelic discrimination assay available from Applied Biosystems may be useful for determining the presence or absence of a variant allele. In a TaqmanB allelic discrimination assay, a specific, fluorescent, dye-labeled probe for each allele is constructed. The probes contain different fluorescent reporter dyes such as FAM and VICTM to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., “3′-minor groove binder-DNA probes increase sequence specificity at PCR extension temperature, “Nucleic Acids Research 28:655-661 (2000)). Minor grove binders include, but are not limited to, compounds such as dihydrocyclopyrroloindole tripeptide (DPI,).

Sequence analysis also may also be useful for determining the presence or absence of a variant allele or haplotype.

Restriction fragment length polymorphism (RFLP) analysis may also be useful for determining the presence or absence of a particular allele (Jarcho et al. in Dracopoli et al., Current Protocols in Human Genetics pages 2.7.1-2.7.5, John Wiley & Sons, New York; Innis et al.,(Ed.), PCR Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (Mullis et al., supra, (1994)). One skilled in the art understands that the one or more nucleotide mismatches that distinguish between the disease-predisposing allele and one or more other alleles arc preferably located in the center of an allele-specific oligonucleotide primer to be used in allele-specific oligonucleotide hybridization. in contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well known assay that may be used to detect a SNP or a haplotype. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261 (1993); White et al., Genomics 12:301-306 (1992)).

The technique of single strand conformational, polymorphism (SSCP) also may be used to detect the presence or absence of a SNP and/or a haplotype (see Hayashi, K., Methods Applic. 1:34-38 (1991)). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a SNP and/or a haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

Other molecular methods useful for determining the presence or absence of a SNP and/or a haplotype are known in the art and useful in the methods of the invention. Other well-known approaches for determining the presence or absence of a SNP and/or a haplotype include automated sequencing and RNAase mismatch techniques (Winter et al., Proc. Natl. Acad. Sci. 82:7575-7579 (1985)). Furthermore, one skilled in the art understands that, where the presence or absence of multiple alleles or haplotype(s) is to be determined, individual alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple alleles can be detected in individual reactions or in a single reaction (a “multiplex” assay). In view of the above, one skilled in the art realizes that the methods of the present invention for diagnosing or predicting susceptibility to or protection against CD in an individual may be practiced using one or any combination of the well known assays described above or another art-recognized genetic assay.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Generally

Previous evidence has shown that the IL23-IL17 pathway is important in pathogenesis of Crohn's disease (CD) and that IL23-IL17 pathway genes including IL12b, IL12RB1, IL12RB2, IL17A, IL17RA are associated with CD. IL17RD, another member of the IL17 receptor family, has been detected in various cells, but its role in human CD has been previously unclear. The inventors determined whether IL17RD is associated with CD and whether there is a gene-gene interaction within IL23-IL17 pathway genes. 763 CD subjects and 254 controls were genotyped for single nucleotide polymorphisms in the IL23A, IL23R, IL17A, IL17RA, IL12B, IL12RB1, IL12RB2 and IL17RD genes using Illumina and ABI platforms. Haplotypes were assigned using Phase v2 and were tested for association with CD by chi square test. The inventors utilized multidimensionality reduction (MDR) to explore gene-gene interactions. Two Blocks (B) of IL17RD were found to he associated with CD. CD patients had a higher frequency of haplotype2 in block2 (B2H2, 55.0% vs. 45.4%, OR=1.5. p=0.01) and a lower frequency of B1H2 (39.1% vs. 50.2%, OR=0.64, p=0.002) and B2113 (37.8% vs. 47.4%, OR=0.68, p=0.01) when compared with controls. Haplotypes with increased risk for CD were observed in the IL23R_B2H1 and B3H1, IL17A_H2, IL17RA_B2H4, IL12RB1_H1 and IL12RB2_H3; haplotypes with decreased risk were observed in the IL23R_B2H2 and B3H2, IL17A_H4, IL17RA_B1H3, IL12B_H1 and IL12RB2_H4. MDR analysis suggested interaction between IL23R_B2H2, IL12RB2_H4 and IL17RD_B2H2 (CV consistency 10/10, tested accuracy 59.7%, p=0.002). The following logistic regression analysis confirmed the interaction (IL23R_B2H2*IL12RB2_H4, p<0.0001; IL23R_B2H2*IL17RD_B2H2, p=0.02). Thus, the inventors have shown that IL17RD is significantly associated with CD and is likely to interact with IL23R in the risk of developing CD.

### Example 2

### Negative Association of IL17-IL23 Pathway—Related SNPs with Crohn's Disease: Table 1

TABLE 1

| | | | | | Percent with | |
|---|---|---|---|---|---|---|
| | | | | | Minor Allele | |
| | | | | TaqMan Assay | Controls | CD |
| dbSNP | Gene(s) | Chr | Position | (if used) | N = 257 | N = 753 |
| rs475825 | IL12A, p35 | 3 | 161,193,022 | C_2936113_10 | 29.3 | 32.9 |
| rs583911 | IL12A, p35 | 3 | 161,193,084 | C_2936112_10 | 71.0 | 67.2 |
| rs2243130 | IL12A, p35 | 3 | 161,193,686 | C_2936111_10 | 7.0 | 11.0 |
| rs2243149 | IL12A, p35 | 3 | 161,198,406 | C_2936107_10 | 64.7 | 63.7 |
| rs2254073 | IL17C | 16 | 87,233,305 | | 27.6 | 25.3 |
| rs7985552 | IL17D | 13 | 20,178,738 | | 20.6 | 19.1 |
| rs6490604 | IL17D | 13 | 20,182,828 | | 26.4 | 24.6 |
| rs9579932 | IL17D | 13 | 20,188,644 | | 11.0 | 10.7 |
| rs7787 | IL17D | 13 | 20,195.198 | | 50.4 | 48.9 |
| rs721430 | IL17F | 6 | 52,210,599 | | 35.1 | 37.1 |
| rs11465551 | IL17F | 6 | 52,211,823 | | 7.7 | 8.6 |
| rs7771511 | IL17F | 6 | 52,212,141 | | 3.5 | 4.9 |
| rsl 2201582 | IL17F | 6 | 52,212,648 | | 18.2 | 17.4 |
| rs12203582 | IL17F | 6 | 52,213.516 | | 42.4 | 40.3 |
| rs1266828 | IL17F | 6 | 52,216,021 | | 25.5 | 24.9 |
| rs455863 | IL17RE/IL17RC | 3 | 9,931,279 | | 48.1 | 47.5 |
| rs8883 | IL17RE/IL17RC | 3 | 9,932,898 | | 48.1 | 46.8 |
| rs4686383 | IL17RE/IL17RC | 3 | 9.934.713 | | 17.7 | 18.5 |
| rs708567 | IL17RE/IL17RC | 3 | 9,935,070 | | 48.1 | 47.4 |
| rs7627880 | IL17RE/IL17RC | 3 | 9,944,328 | | 45.7 | 45.9 |
| rs279545 | IL17RE/IL17RC | 3 | 9,947,494 | | 19.0 | 20.0 |
| rs11171806 | IL23A, p19 | 5 | 55,019,798 | C_25985467_10 | 10.7 | 10.8 |

### Example 3

### Synergistic interaction between IL23R and IL17RA: Table 2

TABLE 2
Interaction between IL23R risk haplotypes and IL17RA risk haplotype in all subjects.

| Presence of | | | | | | | |
|---|---|---|---|---|---|---|---|
| IL23R Block 2 | | | | | 95% | Mantel- | |
| HI or IL23R | Presence of | | | Odds | Confidence | Haenszel | Interaction |
| Block 3 HI | IL17RA H4 | CD | Control | Ratio | Interval | P value | P value |
| No | No | 175 | 78 | 1 | | | |
| No | Yes | 65 | 27 | 1.1 | 0.6-1.8 | 0.0003 | 0.036 |
| Yes | No | 370 | 126 | 1.3 | 0.9-1.8 | | |
| Yes | Yes | 138 | 20 | 3.0 | 1.8-5.2 | | |

### Example 4

Interaction between IL23R, IL12RB2, and IL17RD Multifactor dimensionality analysis (MDR):

Table 3

TABLE 3

| | balanced | CV | |
|---|---|---|---|
| Model | accuracy | consistency | P |
| IL23R Block 2 Haplotype 2 | 0.4902 | 4/10 | ns |
| IL23R B2H2, IL12RB2 Haplotype 4 | 0.5667 | 9/10 | 0.06 |
| IL23R B2H2, IL12RB2 H4, IL17RD | 0.5967 | 10/10 | 0.002 |
| Block 2 H2 | | | |

### Example 5

### Interaction Between IL23R, IL12RB2, and IL17RD—Further Test of MDR Model by Logistic regression: Table 4

TABLE 4

| parameter | estimate | P |
|---|---|---|
| IL23R Block 2 Haplotype 2 | −1.17 | <0.0001 |
| IL12RB2 Haplotype 4 | −1.35 | <0.0001 |
| IL17RD Block 2 Haplotype 2 | −1 | NS |
| IL23R B2H2 * IL12RB2 H4 | 1.49 | <0.0001 |
| IL23R B2H2 * IL17RD B2H2 | 0.75 | 0.02 |

While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

1. A method for diagnosing susceptibility to Crohn's disease in an individual, comprising:
determining the presence or absence of a risk haplotype at the IL17RD genetic locus in the individual; and
diagnosing susceptiblity to Crohn's disease in the individual based upon the presence of the risk haplotype at the IL17RD genetic locus.

2. The method of claim 1, wherein the risk haplotype at the IL17RD genetic locus comprises IL17RD Block 2 Haplotype 2.

3. The method of claim 1, wherein the risk haplotype at the IL17RD genetic locus comprises SEQ. ID. NO.: 1, SEQ. ID. NO.: 2 and/or SEQ. 11). NO.: 3.

4. A method for diagnosing susceptibility to Crohn's disease in an individual, comprising:
obtaining a sample from the individual;
assaying the sample to determine the presence or absence of a risk haplotype at the IL17RD genetic locus in the individual; and
diagnosing susceptibility to Crohn's Disease in the individual based upon the presence of the risk haplotype at the IL17RD genetic locus in the sample.

5. The method of claim 4, wherein the risk haplotype at the IL17RD genetic locus comprises IL17RD Block 2 Haplotype 2.

6. The method of claim 4, wherein assaying the sample comprises genotyping for one or more single nucleotide polymorphisms.

7. A method of determining a low probability of developing Crohn's disease in an individual, relative to a healthy subject, comprising:
obtaining a sample from the individual;
assaying the sample to determine the presence or absence of one or more protective haplotypes at the IL17RD genetic locus in the individual; and
diagnosing a low probability of developing Crohn's disease in the individual, relative to a healthy subject, based upon the presence of one or more protective haplotypes at the IL17RD genetic locus.

8. The method of claim 7, wherein the one or more protective haplotypes at the IL17RD genetic locus comprises IL17RD Block 1 Haplotype 2 and/or IL17RD Block 2 Haplotype 3.

9. The method of claim 7, wherein the one or more protective haplotypes at the IL17RD genetic locus comprises SEQ. ID. NO.: 4, SEQ. ID. NO.: 5, SEQ. ID. NO.: 6, SEQ. ID. NO.: 7, SEQ. ID. NO.: 8, and/or SEQ. ID. NO.: 9.

10. The method of claim 7, wherein the one or more protective haplotypes at the IL17RD genetic locus comprises SEQ. ID. NO.: 1, SEQ. ID. NO.: 2 and/or SEQ. ID. NO.: 3.

11. The method of claim 7, wherein assaying the sample comprises genotyping for one or more single nucleotide polymorphisms.

12. The method of claim 7, wherein assaying the sample comprises specific hybridization of genomic DNA to arrayed probes.

13. A method of diagnosing susceptibility to Crohn's disease in an individual, comprising:
obtaining a sample from the individual;
assaying the sample for the presence or absence in the individual of a risk haplotype at the IL17RD genetic locus, a risk haplotype at the IL23R genetic locus, and a risk haplotype at the IL12RB2 genetic locus; and
diagnosing susceptiblity to Crohn's disease in the individual based upon the presence of the risk haplotype at the IL17RD genetic locus, the risk haplotype at the IL23R genetic locus, and the risk haplotype at the IL12R82 genetic locus.

14. The method of claim 13, wherein the risk haplotype at the IL23R genetic locus comprises IL23R Block 2 Haplotype 2.

15. The method of claim 13, wherein the risk haplotype at the IL12RB2 genetic locus comprises IL12RB2 Haplotype 4.

16. The method of claim 13, wherein the risk haplotype at the IL12RB2 genetic locus comprises SEQ. ID. NO.: 10, SEQ. ID. NO.: 11 and/or SEQ. ID. NO.: 12.

17. The method of claim 13, wherein assaying the sample comprises performing a whole-genome microarray assay.

18. The method of claim 13, wherein assaying the sample comprises multidimensionality reduction.
